(19) United States
(12) Patent Application Publication
(10) Pub. No.: US 2024/0307572 A1
(43) Pub. Date: Sep. 19, 2024

(54) DISINFECTION UNIT AND SHEET PROCESSING APPARATUS

(71) Applicant: GLORY LTD., Hyogo (JP)

(72) Inventors: Eita TAKASHIMA, Hyogo (JP); Hiroki ISHINO, Hyogo (JP)

(21) Appl. No.: 18/668,326

(22) Filed: May 20, 2024

Related U.S. Application Data
(63) Continuation of application No. PCT/JP2022/040058, filed on Oct. 27, 2022.

(30) Foreign Application Priority Data
Nov. 22, 2021 (JP) 2021-189328

(51) Int. Cl. A61L 2/10; A61L 2/24; A61L 2/26; B65H 5/00

(52) U.S. Cl. CPC A61L 2/10; A61L 2/24; A61L 2/26; B65H 5/00; A61L 2202/11; A61L 2202/14; A61L 2202/26; B65H 2301/5141; B65H 2511/12; B65H 2511/18

(57) ABSTRACT

Provided is a disinfection unit and a sheet processing apparatus that can disinfect sheets without affecting the processing of the sheets. The disinfection unit includes: a disinfection part disposed to face a sheet that is being transported; a jacket through which a refrigerant for cooling the disinfection part passes without being leaked on a way; and a refrigerant supply device configured to supply the refrigerant to the jacket.

### CROSS-REFERENCE TO RELATED APPLICATION

The present application claims priority to, and contains subject matter to, PCT/JP2022/040058, filed on Oct. 27, 2022, and JP2021-189328, filed on Nov. 22, 2021, the entire contents of each of which incorporated herein by reference.

### TECHNICAL FIELD

The present disclosure relates to a disinfection unit and a sheet processing apparatus.

### BACKGROUND ART

Sheet processing apparatuses for processing sheets are in widespread use. In recent years, there has been growing concern about cleanliness of sheets, particularly with regard to the adhesion of bacteria or viruses to sheets.

The influence of bacteria or virus can be removed or reduced by disinfecting the sheets during the processing of sheets. In view of this, it is conceivable to incorporate a device that has a disinfection function in a sheet processing apparatus. However, some of such devices generate heat during the disinfection, and are negatively influenced by that heat. In that case, it is necessary to cool the device. In addition, in order to sufficiently disinfect the sheet, it is necessary to dispose the device at a position sufficiently close to the sheet.

On the other hand, in the related art, sheet processing apparatuses including a detection device are known as disclosed in PTL 1 for example. The detection device of the sheet processing apparatus disclosed in PTL 1 includes an illumination device. Further, this sheet processing apparatus includes a cooling part that supplies cooling air to the illumination device for the purpose of cooling the illumination device that generates heat.

### CITATION LIST

### Patent Literature

PTL 1
   Japanese Patent Application Laid-Open No. 2007-273351

### SUMMARY OF INVENTION

A disinfection unit according to the present disclosure includes: a disinfection part disposed to face a sheet that is being transported; a jacket through which a refrigerant for cooling the disinfection part passes without being leaked on a way; and a refrigerant supply device configured to supply the refrigerant to the jacket.

### BRIEF DESCRIPTION OF DRAWINGS

FIG. 1 is a schematic view of a disinfection unit according to a first embodiment;

FIG. 2 is a sectional view taken along line II-II of FIG. 1;

FIG. 3 is a sectional view taken along line III-III of FIG. 2;

FIG. 4 is a right schematic view of a sheet processing apparatus including the disinfection unit according to the first embodiment;

FIG. 5 is a left schematic view of the sheet processing apparatus including the disinfection unit according to the first embodiment;

FIG. 6 is a schematic view illustrating an internal configuration of the sheet processing apparatus including the disinfection unit according to the first embodiment;

FIG. 7 is a schematic view of a disinfection unit according to a second embodiment;

FIG. 8 is a schematic view of a disinfection unit according to a third embodiment;

FIG. 9 is a schematic plan view of a main part of a sheet processing apparatus;

FIG. 10 is a schematic view illustrating an exemplary device arrangement in the sheet processing apparatus; and

FIG. 11 is a diagram for describing a sheet interval control.

### DESCRIPTION OF EMBODIMENTS

If the cooling part disclosed in PTL 1 is applied as a device for disinfecting sheets, i.e., if cooling air is supplied to a device for disinfecting sheets, the cooling air after making contact with the device may spread to the surrounding region, thus negatively affecting the sheet processing.

The present disclosure provides a disinfection unit and a sheet processing apparatus that can disinfect sheets without affecting the processing of the sheets.

A disinfection unit according to the present disclosure includes: a disinfection part disposed to face a sheet that is being transported; a jacket through which a refrigerant for cooling the disinfection part passes without being leaked on a way; and a refrigerant supply device configured to supply the refrigerant to the jacket.

The disinfection part may include: a substrate; and a plurality of LEDs mounted on the substrate and configured to emit an ultraviolet ray to the sheet.

The jacket may be disposed to cover the substrate.

The refrigerant supply device may include an injection fan configured to inject, to the jacket, air serving as the refrigerant.

The refrigerant supply device may include a suction fan configured to suction, from the jacket, air serving as the refrigerant.

The refrigerant supply device may include: a pump configured to supply, to the jacket, a liquid serving as the refrigerant; and a radiator configured to cool the liquid.

The disinfection part may include: a first disinfection part disposed to face a first surface of the sheet; and a second disinfection part disposed to face a second surface of the sheet. The jacket may include: a first jacket through which the refrigerant for cooling the first disinfection part passes without being leaked on a way; and a second jacket through which the refrigerant for cooling the second disinfection part passes without being leaked on a way. The refrigerant supply device supplies the refrigerant to the first jacket and the second jacket.

The first disinfection part and the second disinfection part may be disposed to face each other through a space through which the sheet passes.

A sheet processing apparatus according to the present disclosure includes: a housing; a conveyor disposed inside the housing and configured to transport a sheet; a disinfection unit disposed inside the housing and configured to disinfect the sheet that is being transported by the conveyor. The disinfection unit may include: a disinfection part facing the sheet; a jacket through which a refrigerant for cooling the disinfection part passes without being leaked on a way; and a refrigerant supply device configured to supply the refrigerant to the jacket.

A direction in which the refrigerant flows through the jacket may be orthogonal to a direction in which the conveyor transports the sheet.

The sheet processing apparatus according to the present disclosure may further include recognition circuitry disposed next to the disinfection unit and configured to recognize the sheet.

The disinfection unit and the conveyor may have a substantially flush flat surface facing the sheet.

The disinfection unit may disinfect the sheet that is being transported by the conveyor and moved.

An intake hole and an exhaust hole may be formed in the housing, and the refrigerant supply device may include a fan configured to supply, to the jacket, air flown into the housing from the intake hole as the refrigerant, and to eject, to outside of the housing from the exhaust hole, the air that has flown through the jacket.

The housing may include: a first side plate where the intake hole is formed; and a second side plate where the exhaust hole is formed, the second side plate being disposed at a position facing the first side plate through the conveyor.

The disinfection unit may be further configured to be pulled out from the housing in a sliding manner together with the conveyor, and inserted into the housing in a sliding manner together with the conveyor. The disinfection unit may be further configured such that an air inlet of the refrigerant supply device is located near the intake hole and that an air outlet of the refrigerant supply device is located near the exhaust hole when the disinfection unit is inserted to the housing.

The disinfection part may comprise: a substrate; and a plurality of LEDs mounted on the substrate and configured to emit an ultraviolet ray to the sheet. The sheet processing apparatus further may include control circuitry configured to determine an LED to be turned on from among the plurality of LEDs, based on at least one of a width of the sheet along a direction orthogonal to a transport direction of the sheet and a relative position of the sheet in the direction orthogonal to the transport direction.

According to the present disclosure, a disinfection unit and a sheet processing apparatus that can disinfect sheets without affecting the processing of the sheets can be provided.

Embodiments of the present disclosure are elaborated below with reference to the drawings. Note that while there are configurations or concepts denoted with counter suffixes such as “first” and “second”, such as “first disinfection part” in the specification, such counter suffixes are merely for convenience of distinguishing a plurality of identical or similar configurations or concepts from each other. Therefore, in some cases a configuration or concept denoted with the counter suffix “first” and a configuration or concept denoted with the counter suffix “second” may be read interchangeably.

In addition, disinfection, as used herein, is the elimination or reduction of the infectivity of bacteria or viruses that may cause disease in humans or other organisms, and is a concept that encompasses sterilization, inactivation, and destruction.

### First Embodiment

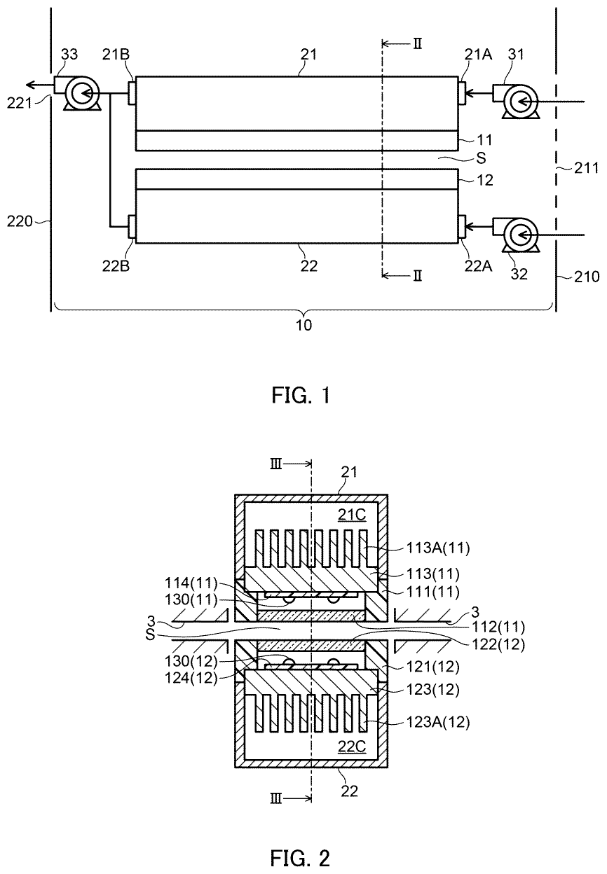

FIG. 1 is a schematic view of a disinfection unit 10 according to the first embodiment. The disinfection unit 10 is disposed inside a sheet processing apparatus, and disinfects sheets processed by the sheet processing apparatus.

Note that the sheet processing apparatus is an apparatus that is installed in a facility such as a financial institution such as a bank, a distribution store such as a convenience store and a security transport institution, and processes sheets such as banknotes and checks, for example. Specific examples of the sheet processing apparatus include banknote coin depositing machines, banknote coin dispensing machines, banknote coin depositing/dispensing machines, automatic teller machines, tax payment machines, money changers, teller machines, ticket vending machines, vending machines, change machines, sales depositing/dispensing machines, banknote counters, banknote sorting machines, banknote bundlers, electronic money charging machines, and banknote disinfection devices.

The disinfection unit 10 includes a first disinfection part 11 and a second disinfection part 12. The first disinfection part 11 and the second disinfection part 12 are disposed to face each other with a space S therebetween.

The disinfection unit 10 includes a first jacket 21 and a second jacket 22. The first jacket 21 is disposed on the back side of the first disinfection part 11, and the second jacket 22 is disposed on the back side of the second disinfection part 12.

The first jacket 21 includes a first jacket inlet 21A and a first jacket outlet 21B. The second jacket 22 includes a second jacket inlet 22A and a second jacket outlet 22B.

The disinfection unit 10 includes a refrigerant supply device that supplies a refrigerant to the first jacket 21 and the second jacket 22. In the first embodiment, the refrigerant supply device includes a first injection fan 31, a second injection fan 32 and a suction fan 33, and supplies air as the refrigerant to the first jacket 21 and the second jacket 22. The arrow in FIG. 1 indicates a flow of the air serving as the refrigerant.

The first injection fan 31 suctions the air flown into the housing through an intake hole 211 formed in a first side plate 210 making up the housing of the sheet processing apparatus where the disinfection unit 10 is disposed, and injects the air into the first jacket 21 through the first jacket inlet 21A. The second injection fan 32 suctions the air flown into the housing through the intake hole 211 formed in the first side plate 210 making up the housing of the sheet processing apparatus where the disinfection unit 10 is disposed, and injects the air into the second jacket 22 through the second jacket inlet 22A.

The suction fan 33 suctions the air in the first jacket 21 through the first jacket outlet 21B. In addition, the suction fan 33 suctions the air in the second jacket 22 through the second jacket outlet 22B. The suction fan 33 ejects the suctioned air to the outside of the housing through an exhaust hole 221 formed in a second side plate 220 making up the housing of the sheet processing apparatus where the disinfection unit 10 is disposed. In FIG. 1, a slight gap is illustrated between the outlet of the suction fan 33 and the second side plate 220 for convenience of description, but the outlet of the suction fan 33 is in air-tight contact with the exhaust hole 221 directly, or through at least one of a duct and a seal. Thus, the air ejected from the suction fan 33 is ejected out of the sheet processing apparatus without being leaked into the housing.

The first injection fan 31 and the second injection fan 32 may be composed of a centrifugal fan, for example. With the first injection fan 31 and the second injection fan 32 composed of centrifugal fans, the air can be relatively quietly supplied. In addition, the suction fan 33 may be composed of an axial fan, for example. With the suction fan 33 composed of an axial fan, a sufficient suction force can be obtained with a relatively compact configuration.

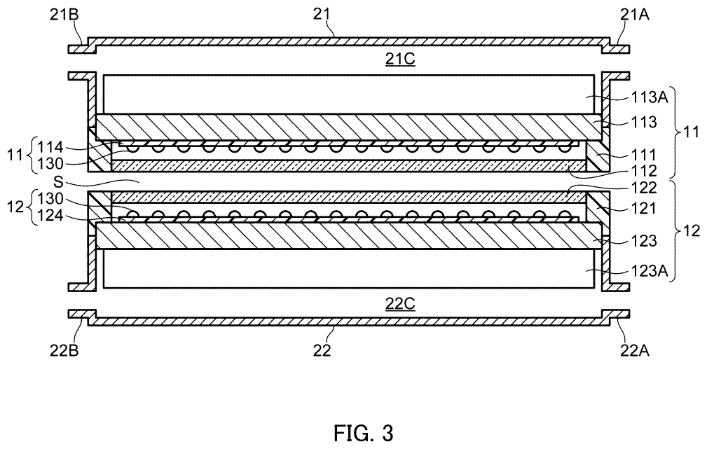

FIG. 2 is a sectional view taken along line II-II of FIG. 1. FIG. 2 illustrates a conveyor 3 provided in the sheet processing apparatus with the first disinfection part 11 and the second disinfection part 12 sandwiched therebetween. In addition, FIG. 3 is a sectional view taken along line III-III of FIG. 2.

The first disinfection part 11 includes a first casing 111, a first window 112, a first heat sink 113 and a first substrate 114. The first window 112 is composed of an elongated rectangular quartz glass plate, and air-tightly attached to an elongated rectangular hole formed on the front side of the first casing 111. Together with the conveyor 3, the first casing 111 and the first window 112 make up a substantially flush flat surface facing the sheet on the space S side.

The first heat sink 113 is formed of a metal with a high thermal conductivity such as aluminum, and attached to the rear side of the first casing 111. A plurality of first fins 113A is formed on the rear side of the first heat sink 113. The shape and structure of the first fin 113A formed at the first heat sink 113 may be appropriately changed or optimized in accordance with the shape of the first jacket 21. For example, it is possible to adopt a structure in which a plate-shaped member connected to an end portion of each of the plurality of first fins 113A and the body of the first heat sink 113 sandwich the first fin 113A.

The first substrate 114 is composed of an elongated rectangular resin plate with substantially the same shape as that of the first window 112, and is disposed in contact with the first heat sink 113 on the front side of the first heat sink 113. On the front side of the first substrate 114, a plurality of LEDs 130 is regularly disposed in staggered two lines in substantially the entire region of the first substrate 114.

The LED 130 is an ultraviolet LED that emits ultraviolet ray when a voltage is applied. The LED 130 is disposed in the immediate vicinity of the first window 112 so as to face the first window 112. Thus, the ultraviolet ray emitted by the LED 130 is emitted with a sufficient intensity to the space S through the first window 112.

The first jacket 21 is disposed on the rear side of the first disinfection part 11. The first jacket 21 is air-tightly attached to the first disinfection part 11, or more specifically, the first casing 111. The first jacket 21 covers the first substrate 114, and in the present embodiment, further covers the first heat sink 113, thus forming a first jacket internal space 21C between it and the first disinfection part 11. The first jacket internal space 21C is a space through which the air serving as the refrigerant passes. The flow direction of the refrigerant in the first jacket 21 and the second jacket 22 is orthogonal to the sheet transport direction of the conveyor 3.

The second disinfection part 12 and the second jacket 22 are configured in the same manner as the first disinfection part 11 and the first jacket 21, respectively. It should be noted that the second disinfection part 12 and the second jacket 22 are symmetric with the first disinfection part 11 and the first jacket 21, respectively with the space S therebetween.

The disinfection unit 10 with the above-described configuration operates as follows. When a voltage is applied to the LED 130, the LED 130 emits an ultraviolet ray. The emitted ultraviolet ray enters the space S through the first window 112 and a second window 122. The sheet (e.g., a banknote) transported by the conveyor 3 passes through the space S from left to right in FIG. 2 during the transport, for example. At this time, the first surface of the sheet faces the first window 112, and is exposed to the ultraviolet ray emitted by the LED 130 of the first disinfection part 11. In addition, the second surface of the sheet faces the second window 122, and is exposed to the ultraviolet ray emitted by the LED 130 of the second disinfection part 12. That is, the both surfaces of the sheet are exposed to the ultraviolet ray. Thus, the both surfaces of the sheet are disinfected. Moreover, since the LED 130 as an ultraviolet ray source is disposed in the immediate vicinity of the space S, i.e., in the immediate vicinity of the sheet passing through the space S, an ultraviolet ray with a sufficient intensity for disinfection can be applied to the sheet. In other words, with this arrangement, the ultraviolet ray can be applied to the sheet without attenuating it on the way in the long path.

The LED 130 emits heat when emitting the ultraviolet ray. This heat causes performance degradation of the LED 130 over time. However, the first substrate 114 and second substrate 124 are in contact with the first heat sink 113 and a second heat sink 123, respectively, and the first heat sink 113 and the second heat sink 123 include the first fin 113A and a second fin 123A, respectively. Further, the first jacket 21 and the second jacket 22 are attached to the first disinfection part 11 and the second disinfection part 12, respectively, and the first jacket 21 and the second jacket 22 are configured to allow the refrigerant to pass therethrough. Thus, the performance degradation of the LED 130 can be prevented or suppressed by efficiently removing the heat from the LED 130.

Moreover, the air flown into the first jacket internal space 21C from the first jacket inlet 21A flows out from the first jacket outlet 21B without being leaked from the joint part of the first disinfection part 11 and the first jacket 21. Likewise, the air flown into a second jacket internal space 22C from the second jacket inlet 22A flows out from the second jacket outlet 22B without being leaked from the joint part of the second disinfection part 12 and the second jacket 22. Naturally, the air is not leaked from the joint part of the first casing 111 and the first window 112, or the joint part of a second casing 121 and the second window 122. Thus, the air flown into the first jacket 21 and the second jacket 22 passes through the first jacket 21 and the second jacket 22 without being leaked on the way.

Thus, the sheet being transported by the conveyor 3 does not make contact with the air serving as the refrigerant. That is, the sheet is not fanned, pushed, or pulled by the air serving as the refrigerant, i.e., not influenced by the air. Thus, as in the case where the disinfection unit 10 is not provided, the sheet processing apparatus including the disinfection unit 10 can transport and process sheets so as to correctly trace a predetermined route while maintaining a predetermined distance between the sheets without causing a jam on the way.

Together with the conveyor 3, the first casing 111 and the first window 112 make up a substantially flush flat surface facing the sheet on the space S side. In addition, together with the conveyor 3, the second casing 121 and the second window 122 make up a substantially flush flat surface facing the sheet on the space S side. With such a step-free arrangement, a sheet jam between the conveyor 3 and the disinfection unit 10 can be more reliably prevented. Note that each of the first casing 111 and the conveyor 3 may include a connecting part with a shape of a plurality of successive irregularities (so-called comb teeth shape). In this case, the connecting parts each include a plurality of recesses and protrusions, and the connecting parts are connected such that the protrusions fit in the recesses of the counterpart. This also applies to the part between the second casing 121 and the conveyor 3. With such connecting parts, the sheet jam between the conveyor 3 and the disinfection unit 10 can be further reliably prevented.

Note that in the disinfection unit 10 according to the first embodiment, the refrigerant supply device includes the first injection fan 31, the second injection fan 32 and the suction fan 33. However, the refrigerant supply device may have different configurations. For example, the refrigerant supply device may include a single injection fan instead of the first injection fan 31 and the second injection fan 32 such that the single injection fan supplies air to the first jacket 21 and the second jacket 22. In addition, the refrigerant supply device may include a first suction fan that suctions air from the first jacket 21 and a second suction fan that suctions air from the second jacket 22 instead of the suction fan 33. In addition, the refrigerant supply device may include only the first injection fan 31 and the second injection fan 32, only a single injection fan, only the suction fan 33, or only the first suction fan and the second suction fan.

In addition, the first jacket outlet 21B and the second jacket inlet 22A may be connected through a pipe. In this case, the air flown into the first jacket 21 through the first jacket inlet 21A flows into the second jacket 22 through the first jacket outlet 21B and the second jacket inlet 22A, and flows out from the second jacket outlet 22B. With such a configuration, the entire configuration of the disinfection unit 10 including the pipe through which the refrigerant flows can be simplified in the case where a sufficient amount of refrigerant for the amount of heat generated from the LED 130 can be supplied.

In addition, in the disinfection unit 10 according to the first embodiment, the first disinfection part 11 and the second disinfection part 12 are disposed to face each other with the space S therebetween. Thus, the disinfection unit 10 can be made compact. It should be noted that the first disinfection part 11 and the second disinfection part 12 may be disposed without facing each other. For example, the first disinfection part 11 and the second disinfection part 12 may be disposed at different positions along the sheet transport direction. Specifically, the second disinfection part 12 may be disposed upstream or downstream of the first disinfection part 11.

Note that the disinfection unit 10 may have a further different configuration. For example, the disinfection unit 10 according to the present embodiment includes two disinfection parts, but the number of disinfection parts may be one, or three or more. In addition, the jacket corresponds to the disinfection unit in a one-to-one relationship. Specifically, the disinfection unit 10 according to the present embodiment includes two jackets, but in the case where one disinfection part or three or more disinfection parts are provided, one jacket or three or more jackets are provided. The refrigerant flowing through one jacket may be configured to cool two or more disinfection units. Other embodiments of the disinfection unit 10 will be described later.

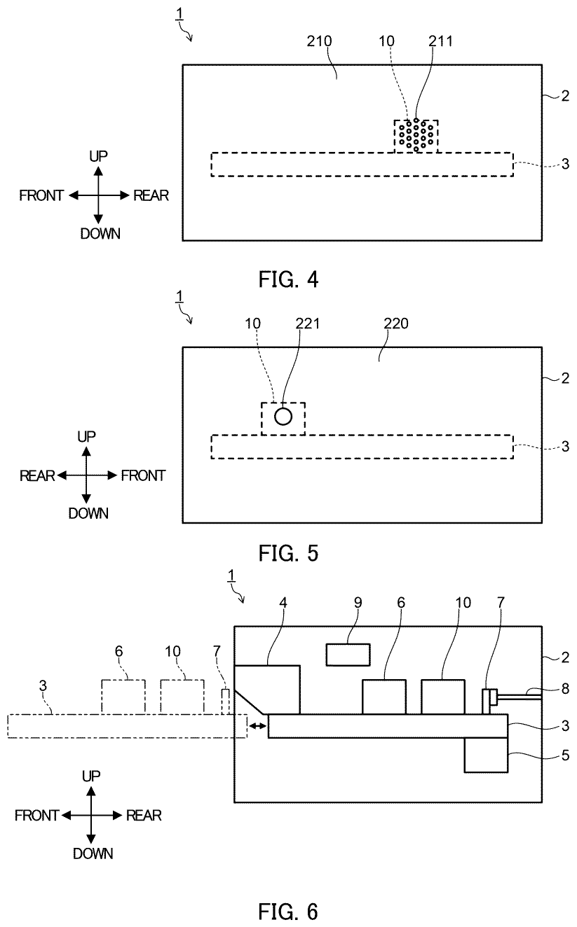

Next, a sheet processing apparatus 1 including the disinfection unit 10 according to the first embodiment is described with reference to FIGS. 4, 5, and 6. Note that in the following description, the front or forward is the side on which the opening inputting sheets to the sheet processing apparatus 1 or the opening for ejecting sheets from the sheet processing apparatus 1 are formed, and the rear or rearward is the opposite side of the front or forward side. In other words, the front or forward is the side on which the user normally using the sheet processing apparatus 1 is located in the sheet processing apparatus 1. In addition, the up or upper side is the side further from the side on which the sheet processing apparatus 1 is installed, and the down or lower side is the opposite side of the up or upper side. In addition, the right or right side is the right side as viewed from the front side of the sheet processing apparatus 1, and the left side is the opposite side of the right or right side.

FIG. 4 is a right schematic view of the sheet processing apparatus 1. The sheet processing apparatus 1 includes a housing 2. The housing 2 includes the first side plate 210 disposed on the right side. The intake hole 211 is formed in the first side plate 210. As indicated with the broken line, the conveyor 3 and the disinfection unit 10 are disposed inside the housing 2. That is, the disinfection unit 10 is disposed at a position overlapping the intake hole 211 in side view. In addition, the first injection fan 31 and the second injection fan 32 are disposed next to the intake hole 211. Thus, there is almost no ventilation resistance between the intake hole 211 and each inlet of the first injection fan 31 and the second injection fan 32. That is, the first injection fan 31 and the second injection fan 32 can efficiently suction the required amount of air from the outside of the housing 2.

Note that there are various mechanical elements inside the housing 2, which may possibility emit heat. That is, the air inside the housing 2 may possibility have a higher temperature than that of the air outside the housing 2. As such, with the configuration in which the disinfection unit 10 is disposed at a position overlapping the intake hole 211 in side view and the first injection fan 31 and the second injection fan 32 are disposed next to the intake hole 211, air with a relatively low temperature can be supplied to the first jacket 21 and the second jacket 22.

FIG. 5 is a left schematic view of the sheet processing apparatus 1. The housing 2 includes the second side plate 220 disposed on the left side. The second side plate 220 is disposed at a position facing the first side plate 210 through the conveyor 3. The exhaust hole 221 is formed in the second side plate 220. The disinfection unit 10 is disposed at a position overlapping the exhaust hole 221 in side view, and the suction fan 33 is disposed next to the exhaust hole 221. Thus, there is almost no ventilation resistance between the outlet of the suction fan 33 and the exhaust hole 221. That is, the suction fan 33 can efficiently eject to the outside of the housing 2 the air inside the first jacket 21 and the second jacket 22.

Note that as described above, the outlet of the suction fan 33 is in air-tight contact with the exhaust hole 221 directly or through at least one of a duct and a seal. Thus, the air ejected from the suction fan 33 is ejected out of the sheet processing apparatus 1 without being leaked into the housing 2.

FIG. 6 is a schematic view illustrating an internal configuration of the sheet processing apparatus 1. Note that FIG. 6 illustrates the same sheet processing apparatus 1 as that of FIGS. 4 and 5 although the scale differs from FIGS. 4 and 5 for convenience of illustration. The sheet processing apparatus 1 includes the conveyor 3, a first endpoint 4, a second endpoint 5, recognition circuitry 6, a positioned member 7, a positioning member 8 and control circuitry 9 inside the housing 2, or in a state partially visible from the outside of the housing 2.

The conveyor 3 is a device that transports sheets when the sheets are processed by the sheet processing apparatus 1. The conveyor 3 can be pulled out from the housing 2 in a sliding manner, and can be inserted to the housing 2 in a sliding manner.

In the case where the sheet processing apparatus 1 is a banknote processing apparatus that can perform a depositing process, the first endpoint 4 is a depositing part or a depositing/dispensing part, and the second endpoint 5 is a storing part that stores banknotes. In the case where the sheet processing apparatus 1 is a banknote processing apparatus that can perform a dispensing process, the first endpoint 4 is a dispensing part or a depositing/dispensing part, and the second endpoint 5 is a storing part. The combination of the first endpoint 4 and the second endpoint 5 is not limited to the above-mentioned combination, and, for example, the first endpoint 4 and the second endpoint 5 may be a depositing part and a dispensing part, respectively. In addition, the first endpoint 4 and the second endpoint 5 may be a temporary storage part and a storing part, respectively. Alternatively, in the case where the sheet processing apparatus 1 is a banknote processing apparatus that can perform a reconciliation process, both the first endpoint 4 and the second endpoint 5 may be a storing part. Note that while the sheet processing apparatus 1 illustrated in FIG. 6 includes one first endpoint 4 and one second endpoint 5, the number of first endpoints 4 and second endpoints 5 may each be two or more.

The recognition circuitry 6 is disposed at the conveyor 3. The recognition circuitry 6 acquires a variety of information for each sheet transported along the conveyor 3, and recognizes the sheet based on various viewpoints. In the case where the sheet is a banknote, the recognition circuitry 6 acquires the serial number from the banknote, and recognizes the authentication, denomination and fitness. The recognition circuitry 6 is configured to move in a sliding manner together with the conveyor 3. In the case where the first endpoint 4 is a depositing part or a depositing/dispensing part, the recognition circuitry 6 is disposed between the disinfection unit 10 and the depositing part or the depositing/dispensing part along the sheet transport direction of the conveyor 3. That is, a sheet (banknote) deposited from the depositing part or the depositing/dispensing part is disinfected by the disinfection unit 10 after being recognized by the recognition circuitry 6.

The positioned member 7 is fixed to a member that moves in a sliding manner together with the conveyor 3 or the conveyor 3. That is, the positioned member 7 is configured to move in a sliding manner together with the conveyor 3. The positioning member 8 is fixed inside the housing 2. The positioned member 7 and the positioning member 8 are described later.

The control circuitry 9 controls the conveyor 3 and the disinfection unit 10. The control circuitry 9 can adjust the ultraviolet ray application time of the disinfection unit 10 on sheets by controlling the conveyor 3. In the case where the first endpoint 4 and the second endpoint 5 include a mechanical element, the control circuitry 9 may control the first endpoint 4 and the second endpoint 5.

The control circuitry 9 includes one or a plurality of computation processing devices such as a CPU (Central Processing Unit) and a MPU (Micro-Processing Unit) and memories. The computation processing device reads and executes programs stored in the memory. The control circuitry 9 controls the entirety of the sheet processing apparatus 1 by executing the program.

The control circuitry 9 may be an element making up the sheet processing apparatus 1, or a computer provided separately from the sheet processing apparatus 1. That is, the sheet processing apparatus 1 may include the control circuitry 9. In addition, the sheet processing apparatus 1 may be controlled by the control circuitry 9 by being connected to the control circuitry 9 directly in a wireless or wired manner, or connected to the control circuitry 9 through a network.

The disinfection unit 10 is disposed next to the conveyor 3 so as to be able to disinfect the moving sheet being transported by the conveyor 3. The disinfection unit 10 is configured to move in a sliding manner together with the conveyor 3.

The sheet processing apparatus 1 with the above-described configuration processes sheets as follows. When performing the processes (e.g., the depositing process, dispensing process or reconciliation process) on sheets, the control circuitry 9 controls the conveyor 3 to transport sheets from the first endpoint 4 to the second endpoint 5. Then, the sheet passes through the disinfection unit 10 (i.e., the position where it can be disinfected by the disinfection unit 10). At this time, the control circuitry 9 activates the disinfection unit 10. Thus, the sheet being transported by the conveyor 3 is disinfected by the disinfection unit 10.

At this time, the LED 130 emits the ultraviolet ray. In turn, the LED 130 emits heat. This heat is taken away by the air serving as the refrigerant passing through the first jacket 21 and the second jacket 22, and thus the LED 130 is cooled. This air passes through the first jacket 21 and the second jacket 22 without being leaked on the way. Thus, the sheet to be disinfected can smoothly pass through the disinfection unit 10 without being influenced by the air flow.

The intake hole 211 and the exhaust hole 221 are formed in the first side plate 210 and the second side plate 220 facing each other with the conveyor 3 therebetween. That is, the air serving as the refrigerant is laterally drawn and laterally ejected. Thus, the air can be smoothly suctioned and exhausted even in the case where the sheet processing apparatus 1 is disposed under a plate-shaped object such as a desk and a table, i.e., even in the case where the sheet processing apparatus 1 is covered with something from above.

In addition, in some situation the disinfection unit 10 and the like may be pulled out in a sliding manner from the housing 2 for the purpose of maintenance or the like. At this time, the conveyor 3, the recognition circuitry 6 and the positioned member 7 are pulled out together with the disinfection unit 10. In FIG. 6, a pulled-out state is illustrated with the chain double-dashed line. The arrow in FIG. 6 indicates the movement direction of the conveyor 3 during the slide movement.

When the maintenance or the like is completed, the conveyor 3, the recognition circuitry 6, the disinfection unit 10 and the positioned member 7 are inserted in a sliding manner into the housing 2. When these devices are inserted into the housing 2, and the disinfection unit 10 is set at the original position, i.e., the position overlapping the intake hole 211 and the exhaust hole 221 in side view, the positioned member 7 makes contact with the positioning member 8. When the positioned member 7 makes contact with the positioning member 8, the conveyor 3, the recognition circuitry 6, the disinfection unit 10 and the positioned member 7 cannot go any further into the housing 2 and stop. Specifically, the disinfection unit 10 is set at a position where the inlets of the first injection fan 31 and the second injection fan 32 are next to the intake hole 211, and the outlet of the suction fan 33 is next to the exhaust hole 221.

Thus, even when the disinfection unit 10 is pulled out to the outside of the housing 2 for maintenance or the like, the disinfection unit 10 can be set at the same position as before the pull-out, i.e., the position where the air serving as the refrigerant can smoothly flow.

### Second Embodiment

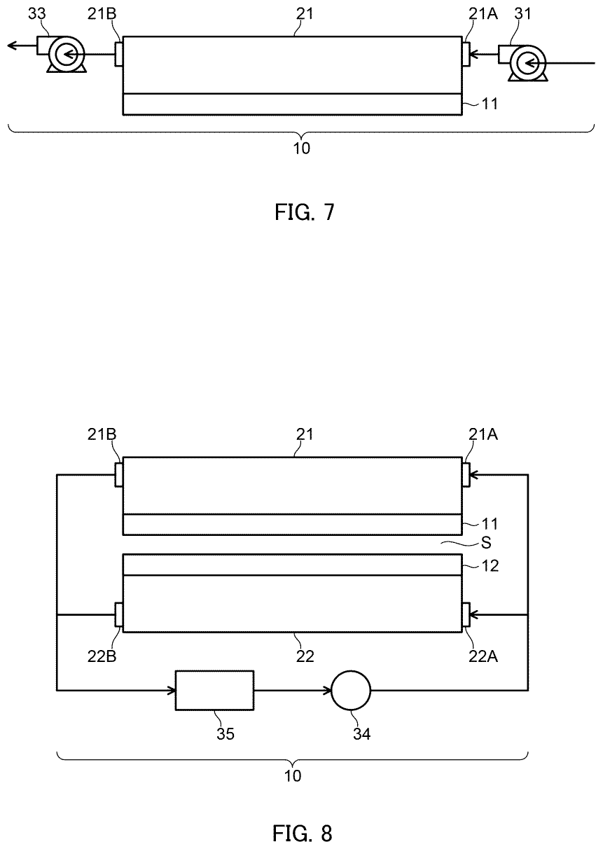

FIG. 7 is a schematic view of the disinfection unit 10 according to a second embodiment. The disinfection unit 10 according to the second embodiment is different from the disinfection unit 10 according to the first embodiment in that it includes one pair of the disinfection part and the jacket instead of two pairs. Specifically, the disinfection unit 10 according to the second embodiment does not include the second disinfection part 12, the second jacket 22 and the second injection fan 32. Other configurations are the same as those of the disinfection unit 10 according to the first embodiment. The first disinfection part 11 is disposed to face one surface of the sheet being transported by the conveyor 3 (see FIG. 6). The arrow in FIG. 7 indicates a flow of air serving as the refrigerant.

In the case where the sheet to be disinfected is a sheet that easily transmits ultraviolet rays, the both surfaces of the sheet can be disinfected with the ultraviolet ray emitted by the first disinfection part 11. In addition, in the case where it is necessary to apply the ultraviolet ray from the both sides of the sheet, the both surfaces of the sheet can be irradiated with the ultraviolet ray by disinfecting the first surface with the disinfection unit 10, and then disinfecting the second surface after inverting the sheet upside down and transporting the sheet to the disinfection unit 10 again.

### Third Embodiment

FIG. 8 is a schematic view of the disinfection unit 10 according to a third embodiment. The disinfection unit 10 according to the third embodiment is different from the disinfection unit 10 according to the first embodiment in the configuration of the refrigerant supply device. The disinfection unit 10 according to the third embodiment uses liquid such as water and ethylene glycol as the refrigerant, for example. Specifically, the refrigerant supply device according to the third embodiment includes a pump 34 and a radiator 35 instead of the first injection fan 31, the second injection fan 32 and the suction fan 33, and uses the liquid serving as the refrigerant by circulating it in a closed circuit. The arrow in FIG. 8 indicates a flow of the liquid serving as the refrigerant.

The pump 34 supplies the liquid to the first jacket 21 and the second jacket 22, and suctions the liquid that has passed through the first jacket 21 and the second jacket 22. The radiator 35 is disposed on the suctioning side or discharging side of the pump 34, and lowers the temperature of liquid that is raised due to the passage through the first jacket 21 and the second jacket 22. Note that the refrigerant supply device according to the present embodiment may further include a tank that stores liquid. At least two or more of the pump 34, the radiator 35 and the tank may be integrated with one another.

The disinfection unit 10 according to the present embodiment can further efficiently cool the LED 130. In addition, the disinfection unit 10 according to the present embodiment can increase the degree of freedom of the arrangement of the disinfection unit 10 inside the sheet processing apparatus because it circulates the refrigerant without taking it from the outside. It is possible to prevent the user from hearing the driving sound of the pump by disposing the pump 34 at a position remote from the user of the sheet processing apparatus such as a position on the rear side and lower side of the sheet processing apparatus 1. That is, quietness can be increased. In addition, by disposing the radiator 35 at a ventilated region inside the housing of the sheet processing apparatus, or a region outside the housing, the temperature of the circulating liquid can be efficiently lowered.

Note that in each of the above-described embodiments, the disinfection unit 10 may include other ultraviolet ray irradiation devices such as ultraviolet ray lamps instead of the LED 130 that ultraviolet rays. In addition, the disinfection unit 10 may include a device such as a heater that disinfects sheets through heating instead of ultraviolet ray irradiation.

### (Lighting Control of LED)

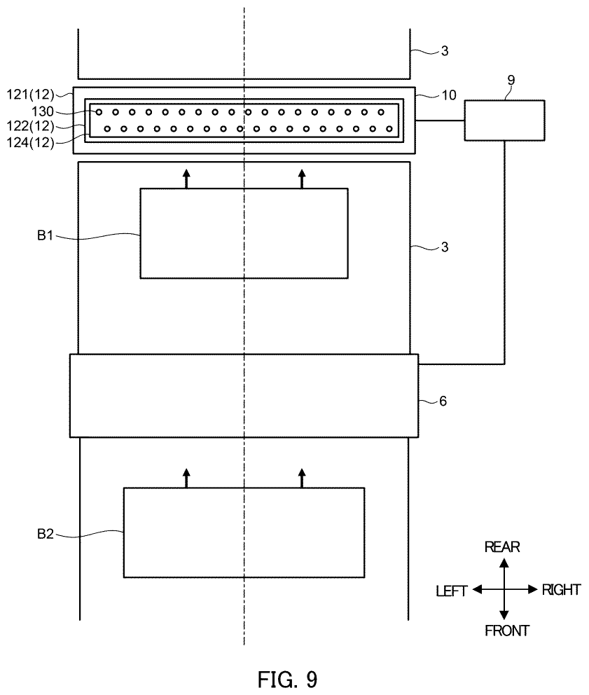

Next, a lighting control of the LED 130 is described with reference to FIG. 9. FIG. 9 is a schematic plan view of a main part of a sheet processing apparatus (e.g., the sheet processing apparatus 1 illustrated in FIGS. 4 to 6). FIG. 9 illustrates the recognition circuitry 6 and the disinfection unit 10 disposed at a position next to the conveyor 3. In addition, FIG. 9 illustrates the control circuitry 9 configured to be able to transmit and receive signals to and from the recognition circuitry 6 and the disinfection unit 10.

While FIG. 9 illustrates only the second disinfection part 12 as a component of the disinfection unit 10, the disinfection unit 10 includes the first disinfection part 11 disposed to face the second disinfection part 12 (see FIGS. 1 to 3). While a lighting control of the LED 130 of the second disinfection part 12 is described below, the LED 130 of the first disinfection part 11 can also be controlled in the same manner.

The dashed line illustrated in FIG. 9 is a center line in plan view of the conveyor 3. The conveyor 3 is configured such that sheets move in a short-edge posture along the center line. Note that the short-edge posture is a posture of a rectangular sheet with the short side aligned along the transport direction and the long side being orthogonal to the transport direction. In addition, the conveyor 3 is configured to transport sheets such that the center of the long side of the sheets is located on the center line of the conveyor 3. In other words, the conveyor 3 is configured to transport sheets in the state where the center of the sheets in the width direction is aligned with the center of the conveyor 3 in the width direction. Note that the conveyor 3 may transport sheets in a long-edge posture. The long-edge posture is a posture of a rectangular sheet with the long side aligned with the transport direction and the short side being orthogonal to the transport direction. Note that the width used herein is the length orthogonal to the sheet transport direction and along the horizontal direction.

FIG. 9 illustrates a first sheet B1 and a second sheet B2 as sheets to be processed. The sheets are transported in the direction indicated with the arrow in FIG. 9, i.e., from the bottom to top in FIG. 9. Specifically, the lower side in FIG. 9 is the upstream side of the sheet transport direction, and the upper side in FIG. 9 is the downstream side of the sheet transport direction. In the following description, the sheet transport direction (the up-down direction in FIG. 9) may be referred to as front-rear direction, and the direction orthogonal to the sheet transport direction and along the surface of sheets being transported (the left-right direction in FIG. 9) may be referred to as left-right direction.

A sheet to be processed is recognized by the recognition circuitry 6 while being transported by the conveyor 3. For example, in the case where the sheet is a banknote, the denomination is recognized. The denomination corresponds to the width (the length in the left-right direction) of a banknote in a one-to-one relationship. Thus, the control circuitry 9 can obtain the width of the sheet on the basis of the recognition result. The recognition circuitry 6 may directly measure the width of the sheet.

Depending on the width of the sheet, the ultraviolet ray emitted by the LEDs 130 disposed near the left and right ends among the LEDs 130 of the second disinfection part 12 may not impinge on the sheet. That is, depending on the width of the sheet, turning on all the LEDs 130 may be a waste of the power consumption in some situation.

In view of this, the control circuitry 9 may control which LED 130 is turned on and which LED 130 is not to be turned in accordance with the width of the sheet. By performing the lighting control of the LED 130 in this manner, the required disinfection can be performed with lower power consumption.

For example, in the example illustrated in FIG. 9, the second sheet B2 has a greater width than the first sheet B1. As such, fewer LEDs 130 may be turned on when disinfecting the first sheet B1 than when disinfecting the second sheet B2.

More specifically, when the first sheet B1 passes above the second disinfection part 12, the ultraviolet rays emitted by five LEDs 130 at the left end and five LEDs 130 at the right end of the second disinfection part 12 do not impinge on the first sheet B1. As such, when disinfecting the first sheet B1, the control circuitry 9 may not turn on the five LEDs 130 at the left end and the five LEDs 130 at the right end of the second disinfection part 12, while turning on the other LEDs.

In addition, when the second sheet B2 passes above the second disinfection part 12, the ultraviolet rays emitted by three LEDs 130 at the left end and three LEDs 130 at the right end of the second disinfection part 12 do not impinge on the second sheet B2. As such, when disinfecting the second sheet B2, the control circuitry 9 may not turn on the three LEDS 130 at the left end and the three LEDs 130 at the right end of the second disinfection part 12, while turning on the other LEDs.

Note that the width of the sheet may be obtained from other sources than the recognition results of the recognition circuitry 6. For example, in the case where the type and width of the sheet to be processed are known in advance, the control circuitry 9 can perform the lighting control of the LED 130 in accordance with the width of the sheet by providing an instruction to the control circuitry 9 in advance.

In addition, the conveyor 3 may be configured to transport sheets in the state where the center of the sheets in the width direction and the center of the width direction of the conveyor 3 do not coincide with each other. In this case, another sensor disposed at the recognition circuitry 6 or the conveyor 3 may acquire the position of the sheet being transported, and the control circuitry 9 may turn on the appropriate LED 130 on the basis of the acquired result. The acquired position of the sheet is a relative position of the sheet in the direction orthogonal to the transport direction, or more specifically the positions of the left and right ends of the sheet.

The greater the width of the sheet with respect to the width of the transport path, the greater the number of light sources that always turn on each time a banknote passes. That is, in the example illustrated in FIG. 9, the smaller the width of the sheet with respect to the width of the transport path, the greater the effect of the lighting control, and in turn the greater the effect of power saving.

### (Another Example of Arrangement Position of Sterilization Unit)

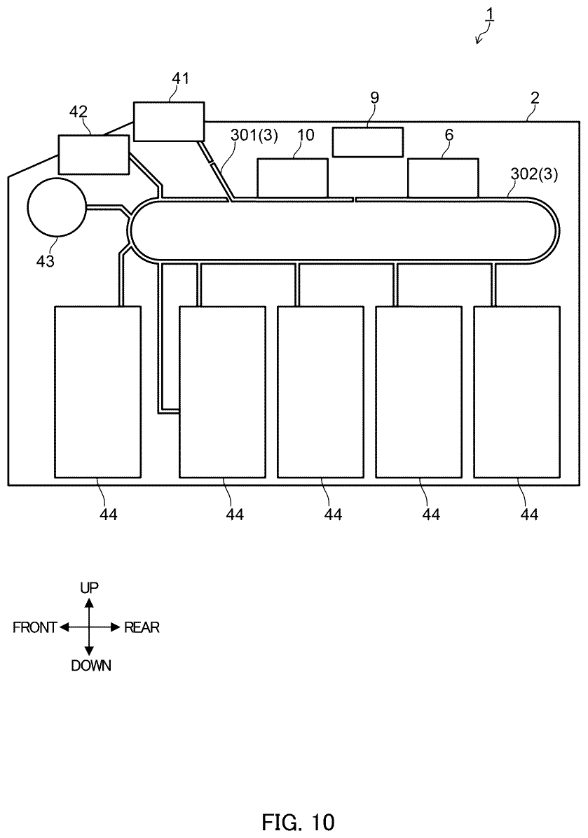

Next, an example of an arrangement of the disinfection unit 10 is described with reference to FIG. 10. The sheet processing apparatus 1 described below is a banknote recycler that processes banknotes serving as sheets. FIG. 10 illustrates an internal structure of the sheet processing apparatus 1 that is a banknote recycler.

The sheet processing apparatus 1 processes banknotes. The sheet processing apparatus 1 includes the housing 2, a depositing part 41, a dispensing part 42, a temporary storage part 43, the conveyor 3, the disinfection unit 10, the recognition circuitry 6, the control circuitry 9, and a plurality of storages 44 disposed inside the housing 2.

The depositing part 41 is a portion for inputting banknotes to be deposited during a depositing process, for example. In addition, the depositing part 41 may be a portion for inputting banknotes to be counted during a counting process, for example. The depositing part 41 includes an inlet. The inlet opens upward at the front part of the housing 2. The operator inputs banknotes by hand to the depositing part 41 through the inlet. The depositing part 41 has a mechanism of taking banknotes one by one into the sheet processing apparatus 1.

The conveyor 3 includes a first conveyor 301 and a second conveyor 302. The first conveyor 301 is disposed between the depositing part 41 and the second conveyor 302. The first conveyor 301 receives banknotes from the depositing part 41, and passes the banknotes to the second conveyor 302. The disinfection unit 10 is disposed at the first conveyor 301.

The second conveyor 2 receives banknotes passed through the first conveyor 301 and transports the banknotes to the destination that is determined by the control circuitry 9 based on the recognition result of the recognition circuitry 6, such as one of the dispensing part 42, the temporary storage part 43, and the plurality of storages 44. The recognition circuitry 6 is disposed at the second conveyor 302.

The first conveyor 301 is configured to transport banknotes at a speed lower than that of the second conveyor 302. For example, the rotational speed of the motor making up the first conveyor 301 is set to be lower than the rotational speed of the motor making up the second conveyor 302.

Thus, the time taken for a single banknote to pass through the disinfection unit 10 can be longer than in the case where the transport speed of the first conveyor 301 is the same as the transport speed of the second conveyor 302. That is, the time for irradiating a single banknote with the ultraviolet ray can be increased. That is, the amount of the ultraviolet ray applied to a single banknote can be increased. In turn, the disinfection effect for each banknotes can be increased.

Note that the banknote transport interval at the first conveyor 301 may be set smaller than the banknote transport interval at the second conveyor 302 by increasing the speed of feeding banknotes by the depositing part 41, i.e., by reducing the interval of passing banknotes to the first conveyor 301 by the depositing part 41. In this manner, the reduction of the banknote processing speed of the sheet processing apparatus 1 can be prevented. The banknote transport interval at the first conveyor 301 and the banknote transport interval at the second conveyor 302 may be controlled by using a detection result of a banknote detection sensor not illustrated in the drawing.

### (Sheet Interval Control)

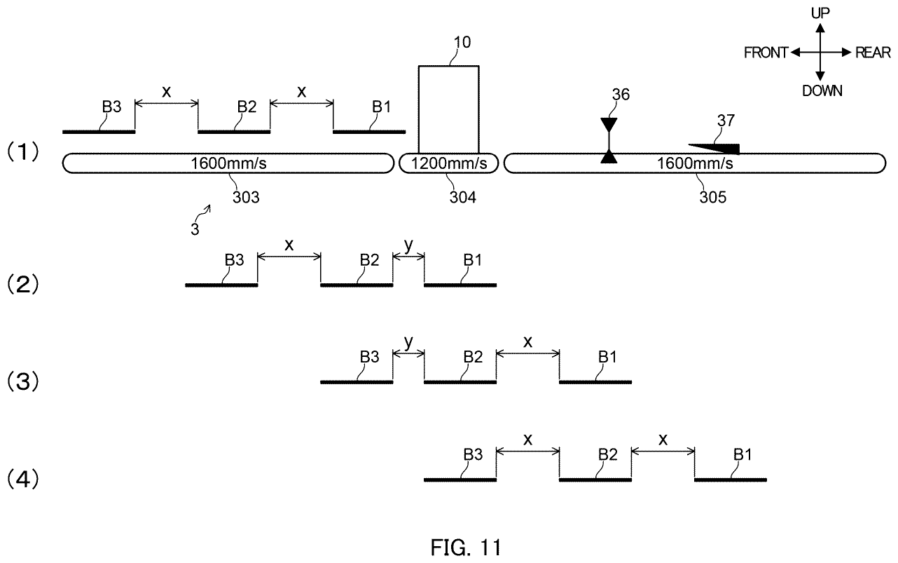

Next, an interval control of sheets is described with reference to FIG. 11. FIG. 11 is a diagram for describing a sheet interval control, and is composed of four parts (1) to (4). (1) to (4) indicate time-series changes in the interval of three sheets.

As illustrated in (1) of FIG. 11, the conveyor 3 includes three parts, i.e., a third conveyor 303, a fourth conveyor 304 and a fifth conveyor 305. Sheets are transported from left to right in FIG. 11.

A recognition circuitry not illustrated in the drawing is disposed at the third conveyor 303. Note that the third conveyor 303 and the fifth conveyor 305 may be connected in a loop form.

The disinfection unit 10 is disposed at the fourth conveyor 304.

A diversion timing sensor 36 and a diversion claw 37 are disposed at the fifth conveyor 305. When a passage of a sheet is detected by the diversion timing sensor 36, the diversion claw 37 is moved to a predetermined position in accordance with the destination of the detected sheet. When the diversion claw 37 is located at a first position, the sheet is directed to a first transport destination by the diversion claw 37, and when the diversion claw 37 is located at the second position, the sheet is directed to a second transport destination by the diversion claw 37. Note that the control of the diversion claw 37 is performed based on the recognition result of the recognition circuitry.

The fourth conveyor 304 has a lower sheet transport speed than the third conveyor 303 and the fifth conveyor 305. For example, the third conveyor 303 and the fifth conveyor 305 transport sheets at 1600 mm/s, and the fourth conveyor 304 transports sheets at 1200 mm/s.

The conveyor 3 with such a configuration transports sheets in the following manner.

First, each sheet is transported by the third conveyor 303 in the state where a distance x is set to the interval between the first sheet B1 and the second sheet B2 and the distance x is set to the interval between the second sheet B2 and the third sheet as illustrated in (1) of FIG. 11.

Subsequently, when the first sheet B1 is passed to the fourth conveyor 304 as illustrated in (2) of FIG. 11, the movement speed of the first sheet B1 decreases. Thus, the interval between the first sheet B1 and the second sheet B2 decreases to a distance y.

Next, when the first sheet B1 is passed to the fifth conveyor 305 and the second sheet B2 is passed to the fourth conveyor 304 as illustrated in (3) of FIG. 11, the movement speed of the first sheet B1 increases and the movement speed of the second sheet B2 decreases. Thus, the interval between the first sheet B1 and the second sheet B2 increases to the distance x. In addition, the interval between the second sheet B2 and third sheet B3 decreases to the distance y.

Further, when the second sheet B2 is passed to the fifth conveyor 305 and the third sheet B3 is passed to the fourth conveyor 304 as illustrated in (4) of FIG. 11, the movement speed of the second sheet B2 increases and the movement speed of the third sheet B3 decreases. Thus, the interval between the second sheet B2 and third sheet B3 increases to the distance x.

By transporting sheets in the above-described manner, the conveyor 3 can suppress the reduction of the total sheet transport speed, i.e., the processing speed as much as possible while reducing the movement speed of sheets passing through the disinfection unit 10. Thus, the time for the disinfection unit 10 to irradiate a single sheet with the ultraviolet ray can be increased while suppressing the reduction of the sheet processing speed as much as possible. That is, the amount of ultraviolet ray for irradiating a single sheet can be increased. In turn, the disinfection effect for each sheet can be increased.

### INDUSTRIAL APPLICABILITY

The present disclosure can be used as sheet processing apparatuses used in the distribution field, financial field, and other various industrial fields, or as disinfection units provided in such sheet processing apparatuses.

1. A disinfection unit, comprising:
a disinfection part disposed to face a sheet that is being transported;
a jacket through which a refrigerant for cooling the disinfection part passes without being leaked on a way; and
a refrigerant supply device configured to supply the refrigerant to the jacket.

2. The disinfection unit according to claim 1, wherein the disinfection part comprises:
a substrate; and
a plurality of LEDs mounted on the substrate and configured to emit an ultraviolet ray to the sheet.

3. The disinfection unit according to claim 2, wherein the jacket is disposed to cover the substrate.

4. The disinfection unit according to claim 1, wherein the refrigerant supply device comprises an injection fan configured to inject, to the jacket, air serving as the refrigerant.

5. The disinfection unit according to claim 1, wherein the refrigerant supply device comprises a suction fan configured to suction, from the jacket, air serving as the refrigerant.

6. The disinfection unit according to claim 1, wherein the refrigerant supply device comprises:
a pump configured to supply, to the jacket, a liquid serving as the refrigerant; and
a radiator configured to cool the liquid.

7. The disinfection unit according to claim 1,
wherein the disinfection part comprises:
a first disinfection part disposed to face a first surface of the sheet; and
a second disinfection part disposed to face a second surface of the sheet,
wherein the jacket comprises:
a first jacket through which the refrigerant for cooling the first disinfection part passes without being leaked on a way; and
a second jacket through which the refrigerant for cooling the second disinfection part passes without being leaked on a way, and
wherein the refrigerant supply device supplies the refrigerant to the first jacket and the second jacket.

8. The disinfection unit according to claim 7, wherein the first disinfection part and the second disinfection part are disposed to face each other through a space through which the sheet passes.

9. A sheet processing apparatus, comprising;
a housing;
a conveyor disposed inside the housing and configured to transport a sheet;
a disinfection unit disposed inside the housing and configured to disinfect the sheet that is being transported by the conveyor,
wherein the disinfection unit comprises:
a disinfection part facing the sheet;
a jacket through which a refrigerant for cooling the disinfection part passes without being leaked on a way; and
a refrigerant supply device configured to supply the refrigerant to the jacket.

10. The sheet processing apparatus according to claim 9, wherein a direction in which the refrigerant flows through the jacket is orthogonal to a direction in which the conveyor transports the sheet.

11. The sheet processing apparatus according to claim 9, further comprising recognition circuitry disposed next to the disinfection unit and configured to recognize the sheet.

12. The sheet processing apparatus according to claim 9, wherein the disinfection unit and the conveyor have a substantially flush flat surface facing the sheet.

13. The sheet processing apparatus according to claim 9, wherein the disinfection unit disinfects the sheet that is being transported by the conveyor and moved.

14. The sheet processing apparatus according to claim 9,
wherein an intake hole and an exhaust hole are formed in the housing, and
wherein the refrigerant supply device comprises a fan configured to supply, to the jacket, air flown into the housing from the intake hole as the refrigerant, and to eject, to outside of the housing from the exhaust hole, the air that has flown through the jacket.

15. The sheet processing apparatus according to claim 14, wherein the housing comprises:
a first side plate where the intake hole is formed; and
a second side plate where the exhaust hole is formed, the second side plate being disposed at a position facing the first side plate through the conveyor.

16. The sheet processing apparatus according to claim 15,
wherein the disinfection unit is further configured to be pulled out from the housing in a sliding manner together with the conveyor, and inserted into the housing in a sliding manner together with the conveyor, and
wherein the disinfection unit is further configured such that an air inlet of the refrigerant supply device is located near the intake hole and that an air outlet of the refrigerant supply device is located near the exhaust hole when the disinfection unit is inserted to the housing.

17. The sheet processing apparatus according to claim 9,
wherein the disinfection part comprises:
a substrate; and
a plurality of LEDs mounted on the substrate and configured to emit an ultraviolet ray to the sheet, and
wherein the sheet processing apparatus further comprises control circuitry configured to determine an LED to be turned on from among the plurality of LEDs, based on at least one of a width of the sheet along a direction orthogonal to a transport direction of the sheet and a relative position of the sheet in the direction orthogonal to the transport direction.
